(19) United States
(12) Patent Application Publication
(10) Pub. No.: US 2008/0228110 A1
(43) Pub. Date: Sep. 18, 2008

(54) Device for computerized dynamic posturography and a method for balance assessment

(76) Inventor: Necip Berme, Worthington, OH (US)

Correspondence Address: KREMBLAS, FOSTER, PHILLIPS & POLLICK, 7632 SLATE RIDGE BOULEVARD, REYNOLDSBURG, OH, 43068, US

(21) Appl. No.: 11/724,482

(22) Filed: Mar. 15, 2007

(51) Int. Cl. A61B 5/00

(52) U.S. Cl. 600/595

(57) ABSTRACT

A device for balance training and assessing dynamic balance by measuring a subject's ability to react to perturbations. A universal joint assembly is translated at the base of a support plate while a top plate on which the subject stands is fixed against translation. The universal joint permits the attached top plate to rotate about at least one and preferably multiple axes, and the subject must control balance following the translation of the universal joint. All components are housed in a one-piece platform assembly, made up of two plates in which the components are mounted. An existing force plate measurement system is placed on the top plate, and the subject stands thereon during use. A virtual environment, by image-creating devices, may be used to create a realistic sensation of tripping and general postural instability, or shifting of the support surface.

### (e) BACKGROUND OF THE INVENTION

1. Field of the Invention

This invention relates generally to a method and apparatus for assessing sensory and motor control impairments.

2. Description of the Related Art

Computerized dynamic posturography provides a means of assessing the underlying sensory and motor control impairments associated with balance disorders. The protocol includes a sensory organizational test where visual, vestibular, and proprioceptive information is manipulated to evaluate their effects on standing balance. The protocol also includes an adaptive motor control test in which a person's ability to recover balance after unexpected perturbations is assessed.

Conventional methods for providing perturbations include moving a platform on which the subject is standing, and pushing or pulling the subject in a controlled fashion. All of these methods involve accelerating and decelerating the subject, which adds a variable to postural control and makes the test results protocol-dependent, as well as acceleration-deceleration profile specific. Therefore, the results are difficult to reproduce, and hence, of limited value. The need exists for an improved means by which to assess a subject's ability to recover balance after unexpected perturbations.

### (f) BRIEF SUMMARY OF THE INVENTION

The invention is a system for determining and training an individual's ability to recover balance when postural instability is introduced by means of a perturbation of the surface supporting the person. The purpose of the invention is to assess and train balance function and stability under the varied conditions encountered in the activities of daily living. The invention is directed at a balance assessment and training system that meets the needs of safety, convenience, and accurate measurement in dynamic testing.

The present invention advantageously provides a means of perturbing the subject's balance without moving the subject or the platform on which the subject stands. The location of the pivot about which the subject balances on the platform is moved relative to the subject and the platform upon which he or she stands. As a result, in order to maintain his/her balance, the subject must react and adapt to the new pivot position. The invention allows the balance to be perturbed both cyclically and randomly, at different amplitudes and frequencies. Furthermore, it is possible to perturb balance in response to the subject's reactions.

The subject's ability to recover from different applied perturbations gives a measure of his/her capability to maintain balance, and his/her risk of falling. This makes the invention ideal for assessing balance capability, while also providing a means of balance training. A virtual environment can be included in either testing or training to create realistic environmental conditions associated with unsteadiness due to perturbation.

The invention includes a one-piece platform assembly made of two frames, such as plates, with the top plate supported by the bottom plate through a joint mounted therebetween. This joint can be a universal joint, or set of pivots, that allows the top plate to freely pivot, causing rotation of the attached top plate. Some displacement and/or velocity-proportional resistance to this rotation may be introduced by further supporting the top platform with a compliant structure, such as a series of springs, a compliant material and/or dashpots. This way, a subject standing on the platform would feel some resistance to the tilting of the top plate, while needing to actively maintain his/her balance. Transducers are incorporated in or on the platform assembly, capable of measuring both the subject's ground reaction forces and the amount of tilt of the platform. Thus, the center of pressure of the subject as well as the rate at which the platform tilts are determined. The preferred embodiment of this device uses a standard force plate measurement system in conjunction with the platform assembly to measure these forces.

Translation of the top plate of the platform is prevented by a mechanism that does not restrict its tilting action. Also, a drive mechanism is used to shift the position of at least one axis of the universal joint relative to the platform. The drive mechanism can perform the shifting motion in a predetermined manner, randomly, or as a response to the subject's balancing action as measured by the transducer. The ability of the subject to re-balance himself/herself, after perturbation due to the movement of the universal joint, provides a measure of his/her ability to maintain balance, and his/her risk of falling in normal daily living. To enhance the testing conditions, a virtual environment, such as seen through image-creating goggles worn by the individual or a screen, can be used. This virtual environment provides a realistic moving image similar to that which may be experienced when one's balance is perturbed, such as during a trip in an everyday environment. Additionally, the virtual environment can confuse what the person is sensing, such as by projecting an image that is inconsistent with, or even contradictory to, the information sensed by the body.

### (g) BRIEF DESCRIPTION OF THE SEVERAL VIEWS OF THE DRAWINGS

FIG. 1A is an exploded view in perspective illustrating the preferred embodiment of the invention with a conventional force plate, on which the subject stands, placed on top of a top plate.

FIG. 1B is a view in perspective illustrating the preferred universal joint assembly that is mounted between the top plate and a lower plate.

FIG. 2A is a side view illustrating the upper section of the universal joint assembly connected to the top plate.

FIG. 2B is a view in perspective illustrating the upper section of the preferred universal joint assembly.

FIG. 2C is a side view in section illustrating the universal joint assembly through the line 2C-2C of FIG. 2B.

FIG. 3A is a side view illustrating the lower section of the universal joint assembly connected to the lower plate.

FIG. 3B is a view in perspective illustrating the components of the universal joint assembly responsible for the translation of components of the universal joint assembly.

FIG. 4A is a view in perspective illustrating a motion limiter assembly that restricts translation of the top plate during translation of the components of the universal joint assembly.

FIG. 4B is a front view illustrating the top and bottom plates connected by motion limiter assemblies, such as the assembly illustrated in FIG. 4A.

In describing the preferred embodiment of the invention which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose. For example, the word connected or term similar thereto are often used. They are not limited to direct connection, but include connection through other elements where such connection is recognized as being equivalent by those skilled in the art.

### (h) DETAILED DESCRIPTION OF THE INVENTION

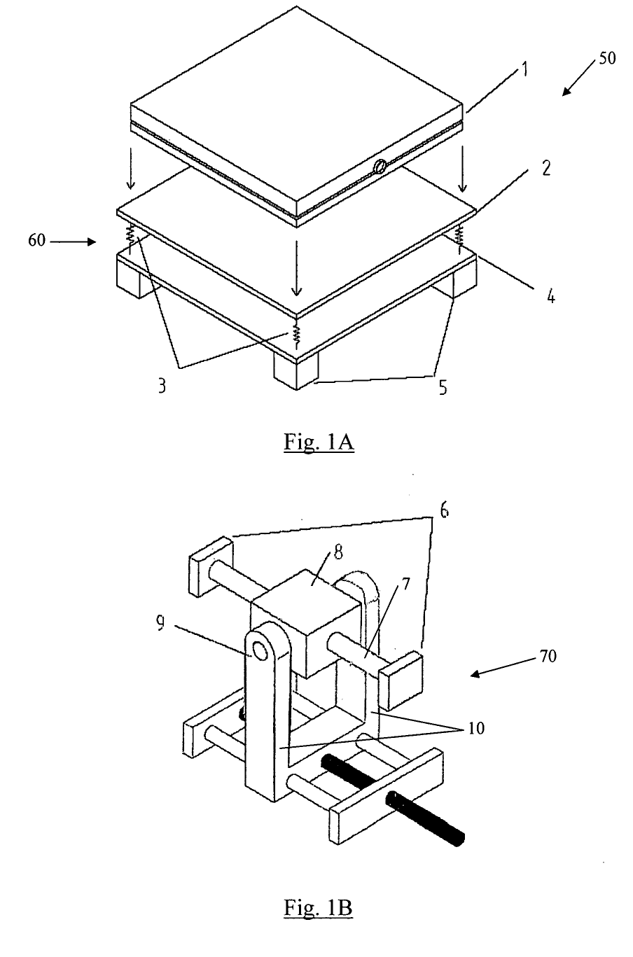

The preferred embodiment of the invention 50 is illustrated in FIG. 1A in association with a conventional force plate measuring device 1. In typical use, the invention includes a conventional safety harness support, which is not illustrated, used to prevent falls; and a computer to which the force plate device 1 and the components discussed below are connected. Thus, the computer can actuate the components below and receive signals from the force plate device 1. The invention 50 is a single assembly for convenience and portability, but which includes several components that are described immediately below in detail.

A platform 60 houses the components of the invention, and the platform includes a first frame, such as the top plate 2, and a second frame, such as the bottom plate 4. The conventional force plate measuring device 1 is preferably mounted to the upper surface of the top plate 2, and the bottom plate 4 is attached to the feet 5, which provide a broad base of stability when the platform is placed on the ground for testing. The term “frame” is intended to include any structure that can provide the type of stability and rigidity that will permit the invention to function as described herein, and includes, but is not limited to, narrow beams and truss-like structures that have holes extending therethrough.

The plates 2 and 4 are rigid, planar panels, and can be made, for example, of stainless steel. Of course, the person of ordinary skill will understand from the description herein that any suitable material, including, without limitation, aluminum, wood or a composite, can be used for the plates 2 and 4. The plates 2 and 4 are preferably oriented substantially parallel to one another with a gap therebetween in the range of a fraction of an inch to 12 inches wide. This dimension is used as an example, and is not intended to be limiting. The gap could be from about one millimeter to several feet wide, depending upon the application circumstances.

The platform 60 has substantially the same width and length as the standard force plate device 1. During use, the feet 5 rest upon the ground with the platform 60 substantially horizontal, and the subject to be tested stands on the top of the force plate device 1 with the bottoms of his or her feet resting on the upper surface of the force plate device 1. The height of the apparatus is preferably no greater than a height that would cause the individual no greater difficulty stepping on the force plate device 1 than taking a step on a typical staircase.

A universal joint assembly 70, illustrated in FIG. 1B, is mounted between the plates 2 and 4, thereby connecting the plates 2 and 4 together as described below, to allow pivoting of the top plate 2 relative to the bottom plate 4. With this configuration, the structural elements that carry out the perturbation of balance are sandwiched between the top plate 2 and the bottom plate 4.

The universal joint assembly 70 allows for the desired perturbation by causing pivoting of the top plate 2 about a pivot axis when the weight of the subject is on the top plate 2 and the pivot point of the assembly 70 is moved relative to the top plate. It will be understood by the person of ordinary skill that any structure that permits relative pivoting along one or more axes between two plates in combination with translation of one or more of the pivot axes relative to the top plate can be substituted for the assembly 70. The assembly 70 is illustrated and described herein as a suitable example of such a structure. Preferably, the assembly has two or more axes of rotation, but one axis is sufficient for a basic embodiment.

As illustrated in FIG. 1B, the universal joint assembly 70 includes a universal joint block 8 that is preferably a block of strong material, such as steel. A bearing passage extends through the block 8, through which a shaft 7 extends slidably and pivotably. Thus, the shaft 7 can pivot about, and translate along the length of, the block 8. In a basic embodiment having only one axis of rotation (not shown), a shaft has a square cross section, and extends through a bearing passage that is also square and of essentially the same dimension, thereby preventing rotation of the shaft relative to the block. This structure provides only one axis of rotation.

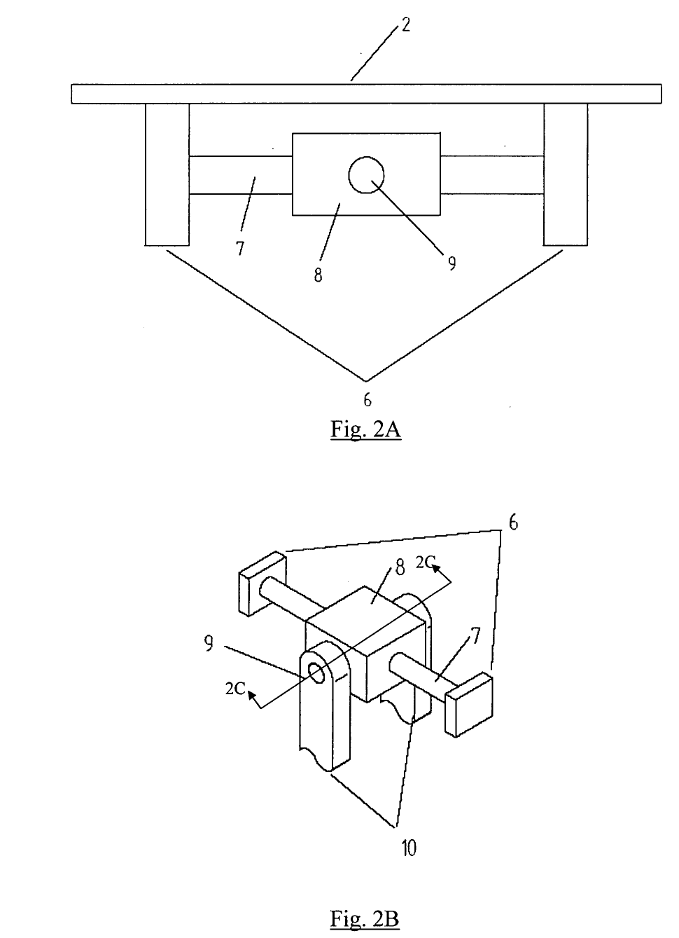

A pair of upper brackets 6 are attached rigidly to the ends of the shaft 7 and fixed at their upper edges to the top plate 2 as shown in FIG. 2A. Thus, the top plate 2 can pivot about the axis of the shaft 7 and translate relative to the block 8 when the shaft 7 slides relative to the block 8.

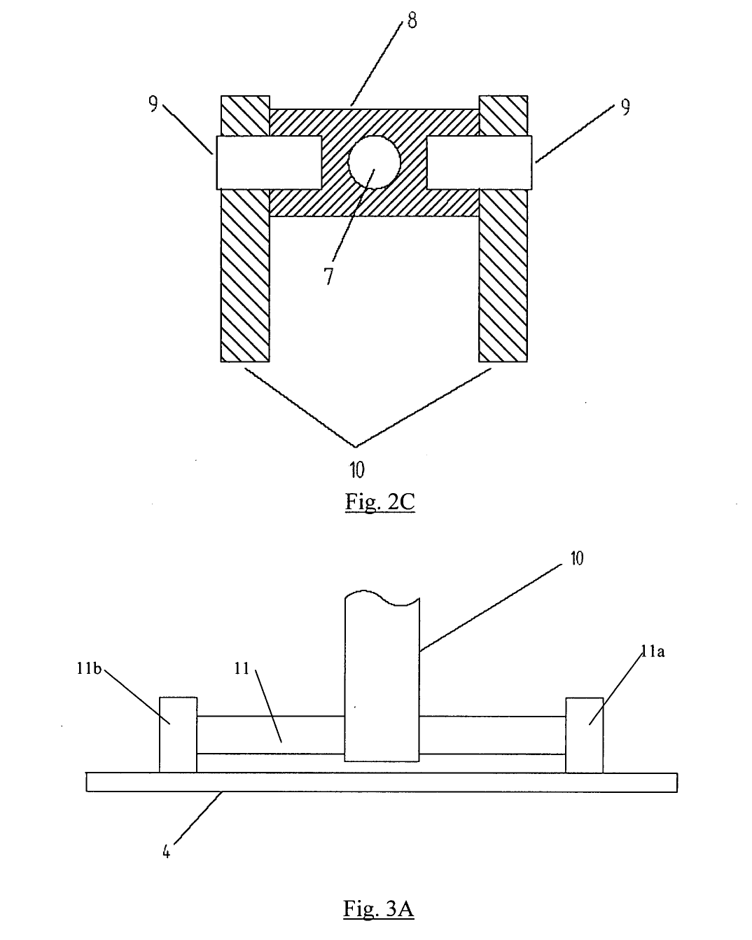

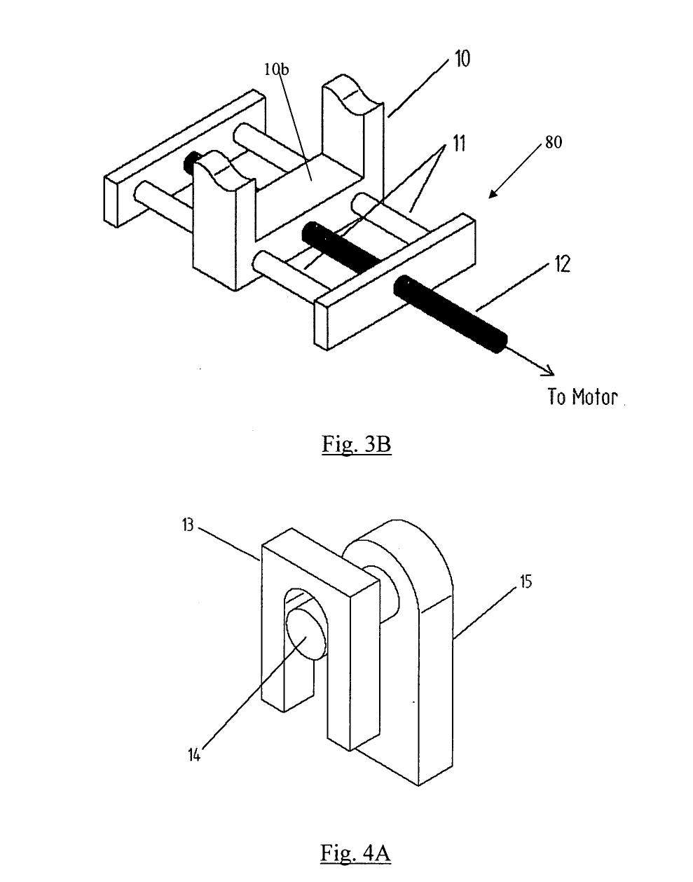

A pair of lower brackets 10 pivotably mount at their upper ends to the block 8 by a pair of short shafts 9 (see FIGS. 2B and 2C). The lower ends of the lower brackets 10 extend to the drive motor assembly 80 as shown in FIG. 3B. The assembly 80 provides translation of at least one of the pivot axes of the universal joint made up of the block 8 and shafts 7 and 9 relative to the upper plate 2. In the case of the assembly 70, the pivot axis translated is the axis of the shafts 9. The translation of the lower brackets 10 is effected by a power-screw 12. The screw 12 is a threaded shaft extending through a threaded passage formed in the connecting beam 10b (see FIG. 3B), and attached at one end to a conventional motor, such as an electric motor, pneumatic motor, or any rotary prime mover (not shown). Two smooth parallel shafts 11 extend through smooth, parallel passages formed in the beam 10b, and the shafts 11 are parallel to the screw 12 to keep the lower brackets from rotating about the screw 12. The beams 11a and 11b are mounted at the ends of the shafts 11, and are rigidly mounted to the lower plate as shown in FIG. 3A. As the power screw 12 rotates, the lower bracket 10 is translated along the pair of lower bracket shafts 11, and this movement translates the universal joint block 8 through the rigid lower brackets 10, relative to the upper and lower plates 2 and 4.

The universal joint block 8 has two axes of rotation: the axes of the shafts 9 and 7. These axes intersect as shown in FIG. 2C, but could be positioned askew, particularly if it is desired for both shafts to translate lengthwise, which is a contemplated alternative. The translation of the pivot axis of the shafts 9 relative to the upper and lower plates 2 and 4 is effected by the drive motor assembly 80 translating the lower brackets 10, which translate the universal joint block 8 relative to the shaft 7. Thus, the top plate 2 has multi-axial rotation (pivoting) relative to the bottom plate 4 about the axes of the shafts 7 and 9. The rotational displacement, which may be a function of the subject's balance characteristics, can be measured by potentiometers or other sensors to determine the angular velocity, acceleration or any other characteristic of the movement. Such sensors are known in the art.

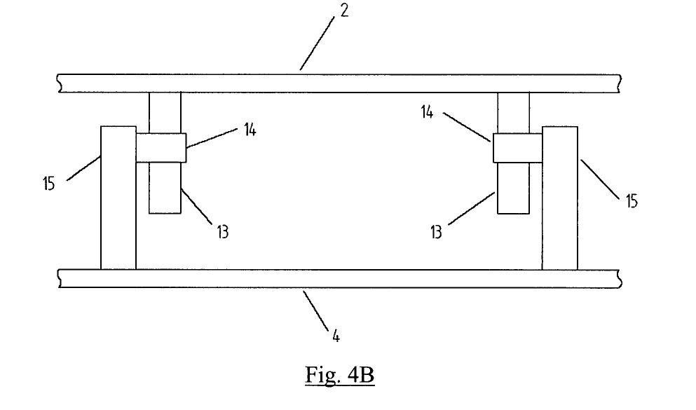

The top plate 2 is maintained in position, when the lower bracket 10 is translated, by a pair of motion limiters shown in FIGS. 4A and 4B. Each motion limiter assembly is comprised of an assembled fork 13, a pivot pin 14, and a pin holder 15. Each pivot pin 14 extends laterally from the upper end of a respective pin holder 15, and the pin holder 15 mounts to, and extends upwardly from, the bottom plate 4. The pin 14 extends through a downwardly facing groove in the fork 13, and the fork 13 mounts to the top plate 2. The assembled fork 13, and attached top plate 2, can therefore rotate about the pivot pin 14, in synch with the rotation of the universal joint block 8 about the shafts 9. Each pivot pin 14 can also slide up and down in its respective groove, allowing the top plate 2 to pivot, thereby matching the multi-dimensional rotational capabilities of the universal joint block 8.

The pins 14 thus prevent any substantial translation of the fork 13 and attached top plate 2 with respect to the pin holder 15, and therefore the bottom plate 4. This means that although the pivot point beneath the individual's feet is translating, as the lower bracket 10 moves the block 8, the top plate 2 on which the individual is standing experiences only multi-axial rotation, and no translation. Preferably the axes of the pivot pins 14 are coaxial with the axis of the shaft 9.

The position of the lower bracket 10 dictates the position of the universal joint, which is at the intersection of the axes of the shafts 7 and 9. Thus, by translating the lower bracket 10, one can thereby effectively translate the universal joint under the subject's feet while preventing translation of the top plate 2 on which the subject's feet rest. The direction and speed of translations can be controlled easily by the computer connected to the motor in order to develop a variety of testing protocols. The translation can also be dictated by the individual's real-time postural sway as detected by transducers in the force plate measurement device 1 and signaled to the computer. This configuration enables a determination of the reaction of the subjects to disruptions in stability. The perturbation is implemented without compromising the safety of the individual or the validity of measurement results, as may be the case in devices which accelerate or decelerate either the patient or the platform surface.

There are preferably compliant structures 3 at spaced positions around the periphery of the assembly 70 between the plates 2 and 4, as illustrated in FIG. 1A. The term “compliant structures” is defined herein to refer to structures that are compressible, such as springs, and structures that dissipate mechanical energy, such as dashpots. This term also includes structures that accomplish both. Examples of compliant structures include, but are not limited to, compressible foam blocks, gas springs, magnetic springs, mechanical springs, elastomeric springs and dashpots. Some compliant structures have the same stiffness and damping rate throughout their useful life. Alternative compliant structures have adjustable stiffness and damping rate, such as by adjusting the pressure and the damping rate of a gas spring used as a compliant structure.

In a preferred embodiment, the compliant structures 3 are mounted to the plates 2 and 4, and therefore remain stationary relative to both plates. In an alternative embodiment (not illustrated), the compliant structures attach only to the top plate, and in another alternative (not illustrated), the compliant structures attach only to the lower plate. Of course, some compliant structures can attach to a combination of the top plate and the lower plate. In these alternative embodiments, the compliant structures remain stationary relative to the plates to which they attach. In another alternative, the compliant structures are mounted to one or more elements of the universal joint so that the compliant structures translate with the translating element of the universal joint. For example, the compliant structures can be mounted to the lower brackets 10 and extend to the edges of the plates to the positions shown in FIG. 1A without attaching to the plates.

The movement of the top plate 2 relative to the lower plate 4 about the universal joint is influenced by the inclusion of compliant structures sandwiched between the top plate 2 and bottom plate 4 of the platform. The compliant structures enable the individual to actively work at controlling their movements for recovery, but attenuate the movements of the plates 2 and 4 relative to one another. The presence of such a structure introduces a feeling that there is a degree of resistance to the movement of the top plate 2 relative to the bottom plate 4, and creates the need to use adaptive balance strategies to regain postural stability.

To measure, compensate, and correct for the inertial forces associated with the movement of the components of the invention, the platform assembly can be instrumented with accelerometers. These will be in addition to, and used in conjunction with, the measurement technologies used to measure the ground reaction forces exerted by the individual standing on the device. All such components are preferably connected to the computer.

Another embodiment of the invention incorporates the components of the standard force plate measuring device directly into the platform assembly. In this alternative embodiment, the invention is used as a stand-alone product with all necessary components, including transducers, within the base of the universal joint platform rather than placing the platform containing the universal joint device directly under an existing force plate for measurement.

Another alternative embodiment allows translation of the universal joint assembly in two directions to increase the degrees of freedom of movement of the pivot location. This multi-axial translation is based on the same principles that drive the translation of the universal joint in the preferred embodiment.

In still another embodiment, a different prime mover is used instead of an electric motor. For example, a pneumatic or hydraulic ram can be used in place of the motor described above. Such substitutes for prime movers are known in the art, and it is impossible to list all such alternatives herein.

With any of the embodiments of the invention, a virtual environment consisting of a realistic moving image, for example viewed through goggles, can be used to enhance testing protocols. This includes, in one embodiment, a pair of glasses worn by the user that projects visible images before the user. Of course, any other means for providing a visible image would suffice, such as a plurality of screens upon which images are projected, a one or more hologram-generating devices, or a hood or other headpiece upon which images can be projected.

The moving image as viewed by the user can be consistent with the sensations of the user through the top plate, such as by causing the image to tilt in the opposite direction that the top plate will tilt when the joint is moved. This gives the consistent sensation of tilting to one side. Alternatively the image can be caused to tilt in the same direction as the top plate, which is inconsistent with the sensations through the top plate. Still further, the image can be caused to stand still, or the image can be caused to tilt or rotate in a direction inconsistent with the tilting of the top plate, such that the user would be confused by the inconsistencies.

The moving image as viewed by the user can be set to tripping mode, in which the images are matched to the center of pressure movements to follow the initial instability and recovery. Alternatively, the image can be set to cyclic motion mode, in which the images follow the cyclic motion of the plate. The inclusion of a virtual environment enhances testing to create more realistic situations that would be encountered in daily activities. This capability will be particularly useful in relation to falls, as by matching the movement of the environment to the center of pressure changes due to perturbation, a situation more closely replicating fall initiation after a trip is possible.

There has been a growing interest in the clinical setting to test an individual's ability to balance under conditions more like those faced in activities of daily living. The invention meets the need for a mechanism that can effectively perturb stance, without interfering with data collection or patient safety. The invention can challenge a subject's stability by quick, unexpected movements of the universal joint, to which the individual must react appropriately to maintain balance. It can also be used to produce cyclic perturbation that brings the subject to his or her threshold of balance maintenance. In both of these modes, center of pressure displacements during recovery, reaction time, and balance strategies are preferably examined, collectively or independently, to determine the abilities of the subject under dynamic conditions. This is used as an indicator of whether the subject is more likely to fall when faced with environmental hazards that cause abrupt changes in postural stability, such as tripping on a rug or walking on uneven ground.

This invention is aimed at clinical applications where analysis of balance is desired, with particular emphasis on identifying subjects with potential problems of postural instability and poor reaction. Aside from assessment of ability, the invention can also be used in balance training and rehabilitation. Although the specific identified need of the invention is in fall prevention, it also allows for a wide variety of other applications, including athletic training and injury recovery. The invention may have particular implications in improving performance in balance and reaction-based physical activities such as dance and gymnastics.

The computer-driven motor to be used in this device allows various protocols to be developed based on specific needs of the physician or rehabilitation specialist. Particular protocols are aimed at translating the universal joint in a sudden, random movement to create a momentary perturbation that must be recovered from. These movements can be at various speeds to elicit different types of reactions from the individual. An additional preferred protocol is one in which the universal joint oscillates continuously, at a given speed and amplitude for each test. The subject's ability to maintain balance as the speed and/or the amplitude of oscillations increase is used as another measure of performance and risk of fall. The invention can also be used in assessing balance in the medio-lateral direction, by having the subject stand facing perpendicular to the direction of the perturbations. Additionally, an embodiment that allows multi-directional translation of the universal joint assembly permits assessment of both the medio-lateral and antero-posterior directions during the same testing procedure.

This detailed description in connection with the drawings is intended principally as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

1. An apparatus upon which a human subject can be disposed for assessing the subject's sensory and motor control, the apparatus comprising:
(a) a first frame;
(b) a second frame disposed beneath the first frame with a gap between the first and second frames;
(c) a joint mounted in the gap between the first and second frames and mounted to the first and second frames, the joint having at least one axis of rotation about which the first frame is mounted to rotate relative to the second frame; and
(d) means for linearly translating said at least one axis of rotation relative to the first frame and the second frame without causing substantial translation of the first frame relative to the second frame.

2. The apparatus in accordance with claim 1, wherein the joint further comprises a second axis of rotation for permitting pivoting movement of the first frame relative to the second frame about said at least one axis of rotation and said second axis of rotation.

3. The apparatus in accordance with claim 1 wherein the joint limits relative movement of the first and second frames to pivoting movement about said at least one axis of rotation.

4. The apparatus in accordance with claim 2 wherein the joint limits relative movement between the first and second frames to pivoting movement about said at least one axis of rotation and said second axis of rotation.

5. The apparatus in accordance with claim 2 wherein the joint further comprises a universal joint.

6. The apparatus in accordance with claim 5 wherein the first and second frames further comprise first and second plates.

7. The apparatus in accordance with claim 6, wherein the universal joint has a first shaft connected to the first plate, and a second shaft mounted to the second plate, and a block through which both shafts extend.

8. The apparatus in accordance with claim 7, wherein the second shaft is longitudinally slidably mounted in the block.

9. The apparatus in accordance with claim 8, wherein said translation means is drivingly linked to the second shaft.

10. The apparatus in accordance with claim 9, further comprising a computer connected to the translation means for actuating the translation means.

11. The apparatus in accordance with claim 10, further comprising image-creating means connected to the computer for producing an image visible to the human eye.

12. The apparatus in accordance with claim 1, further comprising at least one compliance structure mounted in the gap between the first and second frames.

13. The apparatus in accordance with claim 12, wherein the compliance structure is drivingly linked to the translation means for translating the compliance structure when said at least one axis of the joint is translated.

14. The apparatus in accordance with claim 12, further comprising means for adjusting the stiffness and damping rate of the compliance structure.

15. The apparatus in accordance with claim 1, further comprising an integral force plate.

16. A method of assessing a human subject's sensory and motor control, the method comprising:
(a) placing the subject upon a first frame;
(b) disposing a second frame beneath the first frame with a gap between the first and second frames;
(c) mounting a joint to the first and second frames in the gap, the joint having at least one axis of rotation about which the first frame is mounted to rotate relative to the second frame; and
(d) linearly translating said at least one axis of rotation relative to the first frame and the second frame without causing substantial translation of the first frame relative to the second frame.

17. The method in accordance with claim 16, further comprising measuring the subject's response to the translating step.

18. The method in accordance with claim 16, wherein the step of translating further comprises longitudinally sliding a shaft in a bearing.

19. The method in accordance with claim 16, further comprising connecting a computer to at least one of the structures used in the method.

20. The method in accordance with claim 19, further comprising connecting image-creating means to the computer for producing an image visible to the human eye, and disposing the image-creating means in view of the subject.

21. The method in accordance with claim 20, further comprising creating images visible to the subject that are consistent with the translating step.

22. The method in accordance with claim 20, further comprising creating images visible to the subject that are inconsistent with the translating step.

23. The method in accordance with claim 16, further comprising mounting at least one compliance structure in the gap between the first and second frames.

24. The method in accordance with claim 16, further comprising mounting at least one adjustably compliant structure in the gap between the first and second frames, and then adjusting the adjustably compliant structure.

25. The method in accordance with claim 16, wherein the step of translating said at least one axis further comprises translating said at least one axis of the joint a predetermined distance and then observing the subject's response thereto.

26. The method in accordance with claim 16, wherein the step of translating said at least one axis further comprises translating said at least one axis of the joint in a cyclical motion and then observing the subject's response thereto.

27. The method in accordance with claim 16, wherein the step of translating said at least one axis further comprises translating said at least one axis of the joint in a random manner and then observing the subject's response thereto.

28. The method in accordance with claim 16, wherein the step of translating said at least one axis further comprises translating said at least one axis of the joint in a combined random manner and cyclical motion and then observing the subject's response thereto.

29. The method in accordance with claim 17, wherein the step of measuring the subject's response further comprises connecting force sensors to at least one of the frames.

30. The method in accordance with claim 16, further comprising measuring the rotational velocity of the first frame.
